(19) United States
(12) Patent Application Publication
(10) Pub. No.: US 2022/0233674 A1
(43) Pub. Date: Jul. 28, 2022

(54) AN IMMUNOGENIC SEROTYPE 35B PNEUMOCOCCAL POLYSACCHARIDE-PROTEIN CONJUGATE AND CONJUGATION PROCESS FOR MAKING THE SAME

(71) Applicant: Merck Sharp & Dohme Corp., Rahway, NJ (US)

(72) Inventors: Jian He, Blue Bell, PA (US); Patrick McHugh, Pipersville, PA (US); Katherine M. Phillips, Audubon, PA (US); Adriana N. Santiago-Miranda, Conshohocken, PA (US)

(73) Assignee: Merck Sharp & Dohme Corp., Rahway, NJ (US)

(21) Appl. No.: 17/614,865

(22) PCT Filed: Jun. 1, 2020
(86) PCT No.: PCT/US2020/035509

§ 371 Date: Nov. 29, 2021

Related U.S. Application Data
(60) Provisional application No. 62/857,524, filed on Jun. 5, 2019.

(51) Int. Cl. A61K 39/09; A61K 39/385; A61P 31/04

(52) U.S. Cl. CPC A61K 39/092; A61K 2039/6037; A61P 31/04; A61K 39/385

(57) ABSTRACT

The present invention provides a process improvement related to the conjugation of capsular polysaccharides from Streptococcus pneumoniae (S. pneumoniae) serotype 35B to a carrier protein. The serotype 35B polysaccharide-protein conjugate, prepared by the disclosed process, is, among other things, more immunogenic than similar conjugates made by prior art methods. S. pneumoniae serotype 35B polysaccharide-protein conjugates prepared using the processes of the invention can be included in multivalent pneumococcal conjugate vaccine compositions.

### FIELD OF INVENTION

The present invention provides a process improvement related to the conjugation of capsular polysaccharides from Streptococcus pneumoniae (S. pneumoniae) serotype 35B to a carrier protein. The serotype 35B polysaccharide-carrier protein conjugate, prepared by the disclosed process, is, among other things, more immunogenic than similar conjugates made by prior art methods. S. pneumoniae serotype 35B polysaccharide-carrier protein conjugates prepared using the processes of the invention can be included in multivalent pneumococcal conjugate vaccine compositions.

### BACKGROUND OF THE INVENTION

S. pneumoniae, one example of an encapsulated bacterium, is a significant cause of serious disease world-wide. In 1997, the Centers for Disease Control and Prevention (CDC) estimated there were 3,000 cases of pneumococcal meningitis, 50,000 cases of pneumococcal bacteremia, 7,000,000 cases of pneumococcal otitis media and 500,000 cases of pneumococcal pneumonia annually in the United States. See Centers for Disease Control and Prevention, MMWR Morb Mortal Wkly Rep 1997, 46(RR-8):1-13. Furthermore, the complications of these diseases can be significant with some studies reporting up to 8% mortality and 25% neurologic sequelae with pneumococcal meningitis. See Arditi et al., 1998, Pediatrics 102:1087-97.

The multivalent pneumococcal polysaccharide vaccines that have been licensed for many years have proved invaluable in preventing pneumococcal disease in adults, particularly, the elderly and those at high-risk. However, infants and young children respond poorly to unconjugated pneumococcal polysaccharides. Bacterial polysaccharides are T-cell-independent immunogens, eliciting weak or no response in infants. Chemical conjugation of a bacterial polysaccharide immunogen to a carrier protein converts the immune response to a T-cell-dependent one in infants. Diphtheria toxoid (DTx, a chemically detoxified version of DT) and CRM197 have been described as carrier proteins for bacterial polysaccharide immunogens due to the presence of T-cell-stimulating epitopes in their amino acid sequences.

The pneumococcal conjugate vaccine, Prevnar®, containing the 7 most frequently isolated serotypes (4, 6B, 9V, 14, 18C, 19F and 23F) causing invasive pneumococcal disease in young children and infants at the time, was first licensed in the United States in February 2000. Following universal use of Prevnar® in the United States, there has been a significant reduction in invasive pneumococcal disease in children due to the serotypes present in Prevnar®. See Centers for Disease Control and Prevention, MMWR Morb Mortal Wkly Rep 2005, 54(36):893-7. However, there are limitations in serotype coverage with Prevnar® in certain regions of the world and some evidence of certain emerging serotypes in the United States (for example, 19A and others). See O'Brien et al., 2004, Am J Epidemiol 159:634-44; Whitney et al., 2003, N Engl J Med 348:1737-46; Kyaw et al., 2006, N Engl J Med 354:1455-63; Hicks et al., 2007, J Infect Dis 196:1346-54; Traore et al., 2009, Clin Infect Dis 48:S181-S189.

Prevnar 13® is a 13-valent pneumococcal polysaccharide-protein conjugate vaccine including serotypes 1, 3, 4, 5, 6A, 6B, 7F, 9V, 14, 18C, 19A, 19F and 23F. See, e.g., U.S. Patent Application Publication No. US 2006/0228380 A1, Prymula et al., 2006, Lancet 367:740-48 and Kieninger et al., Safety and Immunologic Non-inferiority of 13-valent Pneumococcal Conjugate Vaccine Compared to 7-valent Pneumococcal Conjugate Vaccine Given as a 4-Dose Series in Healthy Infants and Toddlers, presented at the 48^{th} Annual ICAAC/ISDA 46^{th} Annual Meeting, Washington D.C., Oct. 25-28, 2008. See, also, Dagan et al., 1998, Infect Immun. 66: 2093-2098 and Fattom, 1999, Vaccine 17:126. However, there are limitations in serotype coverage with Prevnar 13® in certain regions of the world and some evidence of certain emerging serotypes in the United States, including serotype 35B. See O'Brien et al., 2004, Am J Epidemiol 159:634-44; Whitney et al., 2003, N Engl J Med 348:1737-46; Kyaw et al., 2006, N Engl J Med 354:1455-63; Hicks et al., 2007, J Infect Dis 196:1346-54; Traore et al., 2009, Clin Infect Dis 48:S181-S189; Olarte et al., 2017, J. Clin. Microbiology 55:724-734.

The current multivalent pneumococcal conjugate vaccines have been effective in reducing the incidence of pneumococcal disease associated with those serotypes present in the vaccines. However, the prevalence of the pneumococci expressing serotypes not present in the vaccine has been increasing, as eluded to above. The process conditions for novel serotypes has to be determined for each serotype for conjugation efficiency. Certain serotypes present unique challenges, which include serotype 35B, due to its unique structure. Accordingly, there is a need for an immunogenic serotype 35B polysaccharide-carrier protein conjugate and an improved process for making the same.

### SUMMARY OF THE INVENTION

The present invention provides an immunogenic serotype 35B S. pneumoniae polysaccharide-protein conjugate and a process for preparing the same.

In an embodiment, the invention provides a serotype 35B S. pneumoniae polysaccharide-protein conjugate with a molecular weight of 1,000 kDa to 7,000 kDa.

In another embodiment, the invention provides a serotype 35B S. pneumoniae polysaccharide-protein conjugate with a molecular weight of 1,000 kDa to 7,000 kDa, said conjugate comprising a lysine consumption of 3 mol/mol protein to 9 mol/mol protein.

In another embodiment, the invention provides a serotype 35B S. pneumoniae polysaccharide-protein conjugate with a molecular weight of 1,000 kDa to 7,000 kDa, said conjugate comprising a lysine consumption of 4 mol/mol carrier protein to 8 mol/mol protein.

In an embodiment, the invention provides a serotype 35B S. pneumoniae polysaccharide-protein conjugate with a molecular weight of 1,000 kDa to 5,000 kDa.

In another embodiment, the invention provides a serotype 35B S. pneumoniae polysaccharide-protein conjugate with a molecular weight of 1,000 kDa to 5,000 kDa, said conjugate comprising a lysine consumption of 3 mol/mol protein to 9 mol/mol protein.

In another embodiment, the invention provides a serotype 35B S. pneumoniae polysaccharide-protein conjugate with a molecular weight of 1,000 kDa to 5,000 kDa, said conjugate comprising a lysine consumption of 4 mol/mol carrier protein to 8 mol/mol protein.

In another embodiment, the invention provides a composition comprising a conjugate described above, wherein the composition further comprises free polysaccharide of less than 30% of the total polysaccharide amount and free protein of less than 30% of the total protein amount.

In another embodiment, the invention provides a composition comprising a conjugate described above, wherein the composition further comprises free polysaccharide of less than 20% of the total polysaccharide amount and free protein of less than 20% of the total protein amount.

In another aspect of the conjugate above, the protein of the serotype 35B S. pneumoniae polysaccharide-protein conjugate is CRM197.

In another aspect of the composition above, the protein component of the polysaccharide-protein conjugate is CRM197.

In an embodiment, the invention also provides a process for making a serotype 35B S. pneumoniae polysaccharide-protein conjugate as described above, wherein the process comprises an activation of the polysaccharide wherein the activation utilizes periodate in a range of 0.01 to 0.1 moles of periodate per mole of polysaccharide repeating unit. In another embodiment, the range of periodate is 0.03 to 0.06 moles of periodate per mole of polysaccharide repeating unit.

In another embodiment, the invention provides a process for making an immunogenic serotype 35B S. pneumoniae polysaccharide-protein conjugate as described above, wherein the process comprises conjugating the polysaccharide to the protein, wherein the conjugation is performed at a conjugation temperature of between 22° C. to 38° C. In another embodiment, the conjugation temperature is between 32° C. to 36° C.

In another embodiment, the invention provides a process for preparing a serotype 35B S. pneumoniae polysaccharide-protein conjugate as described above, wherein the process comprises (i) activation of the polysaccharide, wherein the activation utilizes periodate in a range of 0.01 to 0.1 moles of periodate per mole of polysaccharide repeating unit, and (ii) conjugation of the polysaccharide to the protein, wherein the conjugation is performed at a conjugation temperature of between 22° C. to 38° C.

In another embodiment, the invention provides a process wherein the activation of the polysaccharide utilizes periodate in a range of 0.03 to 0.06 moles of sodium metaperiodate per mole of polysaccharide repeating unit.

In another embodiment, the invention provides a process wherein the conjugation temperature is between 32° C. to 36° C.

In another embodiment, the invention provides a multivalent pneumococcal conjugate vaccine composition comprising a serotype 35B S. pneumoniae polysaccharide-protein conjugate as provided in the conjugate embodiments above.

In another embodiment, the invention provides a process for preparing a serotype 35B S. pneumoniae polysaccharide-protein conjugate as described above, wherein the polysaccharide is conjugated to the protein in an aprotic solvent. In a further embodiment, the aprotic solvent is DMSO. In another embodiment, the DMSO solvent comprises less than 1.2% water (v/v). In another embodiment, the DMSO solvent comprises less than 0.6% water (v/v). In another embodiment, the DMSO solvent comprises less than 0.3% water (v/v).

In another embodiment, the invention provides a process for preparing a serotype 35B S. pneumoniae polysaccharide-protein conjugate as described above, wherein the conjugation of the polysaccharide to the protein is conducted in the presence of sodium chloride. In a further embodiment, the concentration of sodium chloride is from about 5 to 15 mM.

### BRIEF DESCRIPTION OF THE DRAWINGS

FIG. 1. Immunogenicity of 35B-CRM197 vaccines, (A) anti-PnPs35B IgG titers (B) anti-35B OPA titers, from eight different formulations. Error bars represent the 95% confidence interval of GMTs.

### DETAILED DESCRIPTION OF THE INVENTION

S. pneumoniae serotype 35B capsular polysaccharide is a complex molecule that contains an activation site in the backbone of the polysaccharide chain. During activation with periodate, the polysaccharide main chain is cleaved as the acyclic triol is oxidized into reactive aldehydes. This results in a decrease in polysaccharide Mw as the number of reactive aldehydes increases, limiting the effective activation range. The terminal aldehydes generated during activation further limit the extent of polysaccharide cross-linking with a carrier protein, resulting in low Mw conjugates.

Due to this structural complexity, efforts to conjugate serotype 35B polysaccharides have been met with limited success when utilizing traditional activation and conjugation processes. For example, activating serotype 35B polysaccharides with standard ranges of periodate used for polysaccharides from other S. pneumoniae serotypes resulted in conjugates that are too small. Additionally, typical Ps:Pr (polysaccharide to carrier protein ratios), polysaccharide concentrations, carrier protein concentrations and temperature ranges used in the conjugation reaction resulted in inadequate conjugate attributes, such as low conjugate Mw, low lysine consumption and high free Ps and carrier protein.

A preferred range of periodate was identified for the activation step that yielded immunogenic serotype 35B polysaccharide-protein conjugates. Activating below the preferred periodate range resulted in polysaccharide-protein conjugates with a desired size but inadequate conjugate attributes, such as low lysine consumption, high free protein and high free polysaccharide, due to the lack of available aldehydes for conjugation. Activating above the preferred periodate range resulted in low Mw conjugates unlikely to be immunogenic, due to the extent of polysaccharide size reduction during the activation reaction.

Further, it was identified that S. pneumoniae serotype 35B polysaccharide is sensitive to the water content in the conjugation reaction, and that the conjugation reaction is likely inhibited. Water interferes with the serotype 35B polysaccharide conjugation reaction by promoting protein and polysaccharide aggregation. In organic conjugation reactions, water can be introduced when spiking additional components, such as salt or reducing agents, into the reaction. The invention presented herein provides a method to reduce water content in the conjugation reaction by eliminating weak reducing agents and including sodium chloride in the pre-lyophilization formulation. In addition, the protein and polysaccharide pre-lyophilization formulations were incorporated together in a co-lyophilization formulation. This eliminated blending of the polysaccharide and protein, allowing conjugation to initialize immediately after dissolution and reducing the absorption of air moisture, further limiting the water content in the reaction.

A preferred temperature range for the S. pneumoniae serotype 35B polysaccharide conjugation reaction was identified that yields immunogenic serotype 35B polysaccharide-protein conjugates with improved conjugate attributes. Conjugating at temperatures below this preferred range resulted in smaller conjugates with inadequate conjugate attributes, notably high free Ps, due to decreased reaction kinetics. Temperatures above the preferred temperature range for the conjugation reaction resulted in poor protein stability and possible protein aggregation.

As used herein, the term “carrier protein” refers to DT (diphtheria toxoid), TT (tetanus toxoid) or CRM197. “Carrier protein” is also referred to as “protein”. In a preferred embodiment, “carrier protein” means CRM197.

As used herein, the term “free protein” means protein present in the composition but not covalently linked to the polysaccharide. The term “conjugated protein” means protein covalently linked to the polysaccharide. The term “total protein” means all protein present in the composition including free protein and conjugated protein.

As used herein, the term “polysaccharide” (Ps) refers to S. pneumoniae capsular polysaccharide. The term “free polysaccharide” means polysaccharide present in the composition but not covalently linked to the carrier protein. The term “conjugated polysaccharide” means polysaccharide covalently linked to the protein. The term “total polysaccharide” means all the polysaccharide present in the composition including free polysaccharide and conjugated polysaccharide.

As used herein, “periodate” includes both periodate and periodic acid. The term also includes both metaperiodate (IO4-) and orthoperiodate (IO6-) and includes the various salts of periodate (e.g., sodium periodate and potassium periodate). In a preferred embodiment, “periodate” refers to sodium metaperiodate.

As used herein, the term “Mw” refers to the weight averaged molecular weight and is typically expressed in Da or kDa. Mw takes into account that a bigger molecule contains more of the total mass of a polymer sample than the smaller molecules do. Mw can be determined by techniques such as static light scattering, small angle neutron scattering, X-ray scattering, and sedimentation velocity.

As used herein, the term “Mn” refers to a number average molecular weight and is typically expressed in Da or kDa. Mn is calculated by taking the total weight of a sample divided by the number of molecules in the sample and can be determined by techniques such as gel permeation chromatography, viscometry via the (Mark-Houwink equation), colligative methods such as vapor pressure osmometry, end-group determination or proton NMR. Mw/Mn reflects polydispersity.

As used herein, the term “Ps:Pr” refers to the mass to mass ratio of polysaccharide to protein in the polysaccharide-protein conjugate. Ps:Pr is directly influenced by the masses of polysaccharide and protein charged into the conjugation reaction. Ps:Pr can be determined by the HPSEC/UV/MALS/RI assay. In an embodiment of the instant invention the Ps:Pr ratio for the serotype 35B polysaccharide-protein conjugate is in the range of 0.5 to 2.0.

As used herein, the term “comprises” when used with the immunogenic composition and/or multivalent pneumococcal conjugate vaccine of the invention refers to the inclusion of any other components (subject to limitations of “consisting of” language for the antigen mixture), such as adjuvants and excipients. The term “consisting of” when used with the multivalent polysaccharide-protein conjugate mixture refers to a mixture having those particular S. pneumoniae polysaccharide protein conjugates and no other S. pneumoniae polysaccharide protein conjugates from a different serotype.

As used herein, the term “activation step” refers to the process of reacting vicinal diols on the serotype 35B S. pneumoniae polysaccharide with an oxidizing agent to form reactive aldehydes.

As used herein, the term “conjugation step” refers to the process of conjugating reactive aldehydes on the serotype 35B S. pneumoniae polysaccharide to lysine groups on the carrier protein, using reductive amination.

Unless otherwise specified, all ranges provided herein are inclusive of the recited lower and upper limits.

### Definitions and Abbreviations

As used throughout the specification and appended claims, the following abbreviations apply:

APA aluminum phosphate adjuvant
CI confidence interval
DMSO dimethyl sulfoxide
GMT geometric mean titer
HPSEC high performance size exclusion chromatography
IM intra-muscular or intra-muscularly
LOS lipo-oligosaccharide
LPS lipopolysaccharide
MALS multi-angle light scattering
MBC monovalent bulk conjugate
Mn number averaged molecular weight
MOPA multiplexed opsonophagocytosis assays
MW molecular weight
NMWCO nominal molecular weight cut off
NZWR New Zealand White rabbit
OPA opsonophagocytosis assay
PCV pneumococcal conjugate vaccine
PD1 post-dose 1
PD2 post-dose 2
PD3 post-dose 3

### PnPs Pneumococcal Polysaccharide

Ps polysaccharide
PS-20 polysorbate-20
RI refractive index
UV ultraviolet
w/v weight per volume

### General Methods for Making Multivalent Pneumococcal Polysaccharide Conjugate Vaccines

### Capsular Polysaccharides

Bacterial capsular polysaccharides, particularly those that have been used as antigens, are suitable for use in the invention and can readily be identified by methods for identifying immunogenic and/or antigenic polysaccharides. Example bacterial capsular polysaccharides from S. pneumoniae are serotypes: 1, 2, 3, 4, 5, 6A, 6B, 6C, 7C, 7F, 8, 9N, 9V, 10A, 11A, 12F, 14, 15A, 15B, 15C, 16F, 17F, 18C, 19A, 19F, 20 (20A and 20B), 22F, 23A, 23B, 23F, 24F, 33F, 35B, 35F, or 38.

Polysaccharides can be purified by known techniques. The invention is not limited to polysaccharides purified from natural sources, however, and the polysaccharides may be obtained by other methods, such as total or partial synthesis. Capsular polysaccharides from S. pneumoniae can be prepared by standard techniques known to those skilled in the art. For example, polysaccharides can be isolated from bacteria and may be sized to some degree by known methods (see, e.g., European Patent Nos. EP497524 and EP497525); and preferably by microfluidization accomplished using a homogenizer or by chemical hydrolysis. S. pneumoniae strains corresponding to each polysaccharide serotype may be grown in a soy-based medium. The individual polysaccharides may then be purified through standard steps including centrifugation, precipitation, and ultrafiltration. See, e.g., U.S. Patent Application Publication No. 2008/0286838 and U.S. Pat. No. 5,847,112. Polysaccharides can be sized in order to reduce viscosity and/or to improve filterability and the lot-to-lot consistency of subsequent conjugated products.

Purified polysaccharides can be chemically activated to introduce functionalities capable of reacting with a carrier protein using standard techniques. The chemical activation of polysaccharides and subsequent conjugation to a carrier protein are achieved by means described in U.S. Pat. Nos. 4,365,170, 4,673,574 and 4,902,506. Briefly, the pneumococcal polysaccharide is reacted with a periodate-based oxidizing agent such as sodium periodate, potassium periodate, or periodic acid resulting in oxidative cleavage of vicinal hydroxyl groups to generate reactive aldehyde groups. Suitable molar equivalents of periodate (e.g., sodium periodate, sodium metaperiodate and the like) include 0.05 to 0.5 molar equivalents (molar ratio of periodate to polysaccharide repeat unit) or 0.1 to 0.5 molar equivalents. The periodate reaction can be varied from 30 minutes to 24 hours depending on the diol conformation (e.g., acyclic diols, cis diols, trans diols), which controls accessibility of the reactive hydroxyl groups to the sodium periodate.

The term “periodate” includes both periodate and periodic acid; the term also includes both metaperiodate (IO⁴⁻) and orthoperiodate (IO⁶⁻) and includes the various salts of periodate (e.g., sodium periodate and potassium periodate). Capsular polysaccharide may be oxidized in the presence of metaperiodate, or in the presence of sodium periodate (NaIO₄). Further, capsular polysaccharide may be oxidized in the presence of orthoperiodate, or in the presence of periodic acid.

Purified polysaccharides can also be connected to a linker. Once activated or connected to a linker, each capsular polysaccharide may be separately conjugated to a carrier protein to form a glycoconjugate. The polysaccharide conjugates may be prepared by known coupling techniques.

Polysaccharide can be coupled to a linker to form a polysaccharide-linker intermediate in which the free terminus of the linker is an ester group. The linker is therefore one in which at least one terminus is an ester group. The other terminus is selected so that it can react with the polysaccharide to form the polysaccharide-linker intermediate.

Polysaccharide can be coupled to a linker using a primary amine group in the polysaccharide. In this case, the linker typically has an ester group at both termini. This allows the coupling to take place by reacting one of the ester groups with the primary amine group in the polysaccharide by nucleophilic acyl substitution. The reaction results in a polysaccharide-linker intermediate in which the polysaccharide is coupled to the linker via an amide linkage. The linker is therefore a bifunctional linker that provides a first ester group for reacting with the primary amine group in the polysaccharide and a second ester group for reacting with the primary amine group in the carrier molecule. A typical linker is adipic acid N-hydroxysuccinimide diester (SIDEA).

The coupling can also take place indirectly, i.e. with an additional linker that is used to derivatize the polysaccharide prior to coupling to the linker.

Polysaccharide can be coupled to the additional linker using a carbonyl group at the reducing terminus of the polysaccharide. This coupling comprises two steps: (a1) reacting the carbonyl group with the additional linker; and (a2) reacting the free terminus of the additional linker with the linker. In these embodiments, the additional linker typically has a primary amine group at both termini, thereby allowing step (a1) to take place by reacting one of the primary amine groups with the carbonyl group in the polysaccharide by reductive amination. A primary amine group is used that is reactive with the carbonyl group in the polysaccharide. Hydrazide or hydroxylamino groups are suitable. The same primary amine group is typically present at both termini of the additional linker which allows for the possibility of polysaccharide (Ps)-Ps coupling. The reaction results in a polysaccharide-additional linker intermediate in which the polysaccharide is coupled to the additional linker via a C—N linkage.

Polysaccharide can be coupled to the additional linker using a different group in the polysaccharide, particularly a carboxyl group. This coupling comprises two steps: (a1) reacting the group with the additional linker; and (a2) reacting the free terminus of the additional linker with the linker. In this case, the additional linker typically has a primary amine group at both termini, thereby allowing step (a1) to take place by reacting one of the primary amine groups with the carboxyl group in the polysaccharide by EDAC activation. A primary amine group is used that is reactive with the EDAC-activated carboxyl group in the polysaccharide. A hydrazide group is suitable. The same primary amine group is typically present at both termini of the additional linker. The reaction results in a polysaccharide-additional linker intermediate in which the polysaccharide is coupled to the additional linker via an amide linkage.

### Carrier Protein

In a particular embodiment of the present invention, CRM197 is used as the carrier protein. CRM197 is a non-toxic variant (i.e., toxoid) of diphtheria toxin. CRM197 may be isolated from cultures of Corynebacterium diphtheria strain C7 (β197) grown in casamino acids and yeast extract-based medium. Further, CRM197 may be prepared recombinantly in accordance with the methods described in U.S. Pat. No. 5,614,382. Typically, CRM₁₉₇ is purified through a combination of ultrafiltration, ammonium sulfate precipitation, and ion-exchange chromatography. In some embodiments, CRM197 is prepared in Pseudomonas fluorescens using Pfenex Expression Technology™ (Pfenex Inc., San Diego, Calif.).

Other suitable carrier proteins include additional inactivated bacterial toxins such as DT (Diphtheria toxoid), TT (tetanus toxoid) or fragment C of TT, pertussis toxoid, cholera toxoid (e.g., as described in International Patent Application Publication No. WO 2004/083251), E. coli LT, E. coli ST, and exotoxin A from Pseudomonas aeruginosa. Bacterial outer membrane proteins such as outer membrane complex c (OMPC), porins, transferrin binding proteins, pneumococcal surface protein A (PspA; See International Application Patent Publication No. WO 02/091998), pneumococcal surface adhesin protein (PsaA), C5a peptidase from Group A or Group B streptococcus, or Haemophilus influenzae protein D, pneumococcal pneumolysin (Kuo et al., 1995, Infect Immun 63; 2706-13) including ply detoxified in some fashion for example dPLY-GMBS (See International Patent Application Publication No. WO 04/081515) or dPLY-formol, PhtX, including PhtA, PhtB, PhtD, PhtE and fusions of Pht proteins for example PhtDE fusions, PhtBE fusions (See International Patent Application Publication Nos. WO 01/98334 and WO 03/54007), can also be used. Other proteins, such as ovalbumin, keyhole limpet hemocyanin (KLH), bovine serum albumin (BSA) or purified protein derivative of tuberculin (PPD), PorB (from N. meningitidis), PD (Haemophilus influenzae protein D; see, e.g., European Patent No. EP 0 594 610 B), or immunologically functional equivalents thereof, synthetic peptides (See European Patent Nos. EP0378881 and EP0427347), heat shock proteins (See International Patent Application Publication Nos. WO 93/17712 and WO 94/03208), pertussis proteins (See International Patent Application Publication No. WO 98/58668 and European Patent No. EP0471177), cytokines, lymphokines, growth factors or hormones (See International Patent Application Publication No. WO 91/01146), artificial proteins comprising multiple human CD4+ T cell epitopes from various pathogen derived antigens (See Falugi et al., 2001, Eur J Immunol 31:3816-3824) such as N19 protein (See Baraldoi et al., 2004, Infect Immun 72:4884-7), iron uptake proteins (See International Patent Application Publication No. WO 01/72337), toxin A or B of C. difficile (See International Patent Publication No. WO 00/61761), and flagellin (See Ben-Yedidia et al., 1998, Immunol Lett 64:9) can also be used as carrier proteins.

Where multivalent vaccines are used, a second carrier can be used for one or more of the antigens in a multivalent vaccine. The second carrier protein is preferably a protein that is non-toxic and non-reactogenic and obtainable in sufficient amount and purity. The second carrier protein is also conjugated or joined with an antigen, e.g., a S. pneumoniae polysaccharide to enhance immunogenicity of the antigen. Carrier proteins should be amenable to standard conjugation procedures. Each capsular polysaccharide not conjugated to a first carrier protein may be conjugated to the same second carrier protein (e.g., each capsular polysaccharide molecule being conjugated to a single carrier protein). Capsular polysaccharides not conjugated to a first carrier protein may be conjugated to two or more carrier proteins (each capsular polysaccharide molecule being conjugated to a single carrier protein). In such embodiments, each capsular polysaccharide of the same serotype is typically conjugated to the same carrier protein. Other DT mutants can be used as the second carrier protein, such as CRM176, CRM228, CRM45 (Uchida et al., 1973, J Biol Chem 218:3838-3844); CRM9, CRM45 CRM102, CRM103 and CRM107 and other mutations described by Nicholls and Youle in Genetically Engineered Toxins, Ed: Frankel, Maecel Dekker Inc, 1992; deletion or mutation of Glu-148 to Asp, Gln or Ser and/or Ala 158 to Gly and other mutations disclosed in U.S. Pat. Nos. 4,709,017 or 4,950,740; mutation of at least one or more residues Lys 516, Lys 526, Phe 530 and/or Lys 534 and other mutations disclosed in U.S. Pat. Nos. 5,917,017 or 6,455,673; or fragment disclosed in U.S. Pat. No. 5,843,711.

### Conjugation by Reductive Amination

Covalent coupling of polysaccharide to carrier protein can be performed via reductive amination in which an amine-reactive moiety on the polysaccharide is directly coupled to primary amine groups (mainly lysine residues) of the protein. As is well known, a reductive amination reaction proceeds via a two step mechanism. First, a Schiff base intermediate, of formula R—CH═N—R′, is formed by reaction of an aldehyde group on molecule 1 (R—CHO) with a primary amine group (R′—NH2) on molecule 2. In the second step, the Schiff base is reduced to form an amino compound of formula R—CH2-NH—R′. While many reducing agents are capable of being utilized, most often a highly selective reducing agent such as sodium cyanoborohydride (NaCNBH₃) is employed since such reagents will specifically reduce only the imine function of the Schiff base.

Since all the polysaccharides have an aldehyde function at the end of the chain (terminal aldehyde function), the conjugation methods comprising a reductive amination of the polysaccharide can be applied very generally and, when there is no other aldehyde function in the repeating unit (intrachain aldehyde function), such methods make it possible to obtain conjugates in which a polysaccharide molecule is coupled to a single molecule of carrier protein.

A typical reducing agent is cyanoborohydride salt such as sodium cyanoborohydride. The imine-selective reducing agent typically employed is sodium cyanoborohydride, although other cyanoborohydride salts can be used including potassium cyanoborohydride. Differences in starting cyanide levels in sodium cyanoborohydride reagent lots and residual cyanide in the conjugation reaction can lead to inconsistent conjugation performance, resulting in variable product attributes, such as conjugate size and conjugate Ps-to-CRM197 ratio. By controlling and/or reducing the free cyanide levels in the final reaction product, conjugation variability can be reduced.

Residual unreacted aldehydes on the polysaccharide are optionally reduced with the addition of a strong reducing agent, such as sodium borohydride. Generally, use of a strong reducing agent is preferred. However, for some polysaccharides, it is preferred to avoid this step. For example, S. pneumoniae serotype 5 contains a ketone group that may react readily with a strong reductant. In this case, it is preferable to bypass the reduction step to protect the antigenic structure of the polysaccharide.

Following conjugation, the polysaccharide-protein conjugates are purified to remove excess conjugation reagents as well as residual free protein and free polysaccharide by one or more of any techniques well known to the skilled artisan, including concentration/diafiltration operations, ultrafiltration, precipitation/elution, column chromatography, and depth filtration. See, e.g., U.S. Pat. No. 6,146,902. In one embodiment, the purifying step is by ultrafiltration.

In an embodiment, the instant invention provides a serotype 35B S. pneumoniae polysaccharide-protein conjugate with a molecular weight of 500 kDa to 10,000 kDa, or 1000 kDa to 10,000 kDa, or 1,000 kDa to 9,000 kDa, or 1,000 kDa to 8,000 kDa, or 1,000 kDa to 7,000 kDa, or 1,000 kDa to 6,000 kDa. Preferably, the instant invention provides a serotype 35B S. pneumoniae polysaccharide-protein conjugate with a molecular weight of 1,000 kDa to 7,000 kDa. Further, preferably, the instant invention provides a serotype 35B S. pneumoniae polysaccharide-protein conjugate with a molecular weight of 1,000 kDa to 5,000 kDa.

In an embodiment, the instant invention provides a process for making a serotype 35B S. pneumoniae polysaccharide-protein conjugate, as noted in the conjugate embodiments above, wherein the process comprises an activation of the polysaccharide wherein the activation utilizes periodate in a range of 0.01 to 0.1 moles of periodate per mole of polysaccharide repeating unit. In another embodiment, the range of periodate is 0.03 to 0.06 moles of periodate per mole of polysaccharide repeating unit.

In another embodiment, the invention provides a process for making an immunogenic serotype 35B S. pneumoniae polysaccharide-protein conjugate, as noted in the conjugate embodiments above, wherein the process comprises conjugating the polysaccharide to the protein, wherein the conjugation is performed at conjugation temperatures of between 22° C. to 38° C. In another embodiment, the conjugation temperature is between 32° C. to 36° C.

In another embodiment, the invention provides a process for preparing a serotype 35B S. pneumoniae polysaccharide-protein conjugate, as noted in the conjugate embodiments above, wherein the process comprises activation of the polysaccharide, wherein the activation utilizes periodate in a range of 0.01 to 0.1 moles of periodate per mole of polysaccharide repeating unit and conjugation of the polysaccharide to the protein wherein the conjugation is performed at a conjugation temperature of between 22° C. to 38° C.

In another embodiment, the invention provides a process wherein the activation of the polysaccharide utilizes periodate in a range of 0.03 to 0.06 moles of sodium metaperiodate per mole of polysaccharide repeating unit.

In another embodiment, the invention provides a process wherein the conjugation temperature is between 32° C. to 36° C.

In an embodiment of the instant invention, an aprotic solvent is used in the conjugation reaction. In another embodiment of the instant invention, DMSO (dimethyl sulfoxide) is used as the aprotic solvent in the conjugation reaction.

In another embodiment, conjugation is conducted in a DMSO solvent with sodium chloride. In another embodiment, the sodium chloride concentration is about 1 mM to 50 mM. In another embodiment, the sodium chloride concentration is about 5 mM to 15 mM.

In another embodiment, conjugation is conducted in a DMSO solvent with less than 1.2% of water (v/v). In another embodiment, the conjugation is conducted in the DMSO solvent with less than 0.6% of water (v/v). In another embodiment, the conjugation is conducted in the DMSO solvent with less than 0.3% of water (v/v).

In an embodiment, the conjugation is conducted in a DMSO solvent using activated polysaccharide with aldehyde per repeat unit in the range of 0.01-0.1. In another embodiment, the conjugation is conducted in the DMSO solvent using activated polysaccharide with aldehyde per repeat unit in the range of 0.03-0.06.

In an embodiment, the conjugation is conducted in a DMSO solvent using activated polysaccharide with molecular weight in the range of 30-200 KDa. In another embodiment, the conjugation is conducted in the DMSO solvent using activated polysaccharide with molecular weight in the range of 40-100 KDa.

In another embodiment, the conjugation reaction is conducted with size reduced polysaccharide made via periodate activation.

### Multivalent Polysaccharide-Protein Conjugate Vaccines

In certain embodiments, the immunogenic compositions can comprise capsular polysaccharides from S. pneumoniae serotype selected from at least one of 1, 2, 3, 4, 5, 6A, 6B, 6C, 7C, 7F, 8, 9N, 9V, 10A, 11A, 12F, 14, 15A, 15B, 15C, 16F, 17F, 18C, 19A, 19F, 20 (20A or 20B), 22F, 23A, 23B, 23F, 24F, 33F, 35B, 35F, or 38 conjugated to one or more carrier proteins. Preferably, saccharides from a particular serotype are not conjugated to more than one carrier protein.

After the individual glycoconjugates are purified, they are compounded to formulate the immunogenic composition of the present invention. These pneumococcal conjugates are prepared by separate processes and bulk formulated into a single dosage formulation.

### Pharmaceutical/Vaccine Compositions

The present invention further provides compositions, including pharmaceutical, immunogenic and vaccine compositions, comprising, consisting essentially of, or alternatively, consisting of any of the polysaccharide serotype combinations described above together with a pharmaceutically acceptable carrier and an adjuvant. The compositions may comprise, consist essentially of, or consist of 2 to 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34 or 35 distinct polysaccharide-protein conjugates, wherein each of the conjugates contains a different capsular polysaccharide conjugated to either the first carrier protein or the second carrier protein, and wherein the capsular polysaccharides from at least one of serotypes 1, 2, 3, 4, 5, 6A, 6B, 6C, 7C, 7F, 8, 9N, 9V, 10A, 11A, 12F, 14, 15A, 15B, 15C, 16F, 17F, 18C, 19A, 19F, 20 (20A or 20B), 22F, 23A, 23B, 23F, 24F, 33F, 35B, 35F, or 38 of Streptococcus pneumoniae are conjugated to CRM197.

In an embodiment, the instant invention provides a multivalent pneumococcal conjugate vaccine comprising a serotype 35B S. pneumoniae polysaccharide-protein conjugate as provided in the conjugate embodiments above.

Formulation of the polysaccharide-protein conjugates can be accomplished using art-recognized methods. For instance, individual pneumococcal conjugates can be formulated with a physiologically acceptable vehicle to prepare the composition. Examples of such vehicles include, but are not limited to, water, buffered saline, polyols (e.g., glycerol, propylene glycol, liquid polyethylene glycol) and dextrose solutions.

In a preferred embodiment, a vaccine composition is formulated in L-histidine buffer with sodium chloride.

As defined herein, an “adjuvant” is a substance that serves to enhance the immunogenicity of an immunogenic composition of the invention. An immune adjuvant may enhance an immune response to an antigen that is weakly immunogenic when administered alone, e.g., inducing no or weak antibody titers or cell-mediated immune response, increase antibody titers to the antigen, and/or lowers the dose of the antigen effective to achieve an immune response in the individual. Thus, adjuvants are often given to boost the immune response and are well known to the skilled artisan. Suitable adjuvants to enhance effectiveness of the composition include, but are not limited to:

(1) aluminum salts (alum), such as aluminum hydroxide, aluminum phosphate, aluminum sulfate, etc.;

(2) oil-in-water emulsion formulations (with or without other specific immunostimulating agents such as muramyl peptides (defined below) or bacterial cell wall components), such as, for example, (a) MF59 (International Patent Application Publication No. WO 90/14837), containing 5% Squalene, 0.5% Tween 80, and 0.5% Span 85 (optionally containing various amounts of MTP-PE) formulated into submicron particles using a microfluidizer such as Model 110Y microfluidizer (Microfluidics, Newton, Mass.), (b) SAF, containing 10% Squalene, 0.4% Tween 80, 5% pluronic-blocked polymer L121, and thr-MDP either microfluidized into a submicron emulsion or vortexed to generate a larger particle size emulsion, (c) Ribi™ adjuvant system (RAS), (Corixa, Hamilton, Mont.) containing 2% Squalene, 0.2% Tween 80, and one or more bacterial cell wall components from the group consisting of 3-O-deacylated monophosphoryl lipid A (MPL™) described in U.S. Pat. No. 4,912,094, trehalose dimycolate (TDM), and cell wall skeleton (CWS), preferably MPL+CWS (Detox™); and (d) a Montanide ISA;

(3) saponin adjuvants, such as Quil A or STIMULON™ QS-21 (Antigenics, Framingham, Mass.) (see, e.g., U.S. Pat. No. 5,057,540) may be used or particles generated therefrom such as ISCOM (immunostimulating complexes formed by the combination of cholesterol, saponin, phospholipid, and amphipathic proteins) and Iscomatrix® (having essentially the same structure as an ISCOM but without the protein);

(4) bacterial lipopolysaccharides, synthetic lipid A analogs such as aminoalkyl glucosamine phosphate compounds (AGP), or derivatives or analogs thereof, which are available from Corixa, and which are described in U.S. Pat. No. 6,113,918; one such AGP is 2-[(R)-3-tetradecanoyloxytetradecanoylamino]ethyl 2-Deoxy-4-O-phosphono-3-O—[(R)-3-tetradecanoyloxytetradecanoyl]-2-[(R)-3-tetradecanoyloxytetradecanoylamino]-b-D-glucopyranoside, which is also known as 529 (formerly known as RC529), which is formulated as an aqueous form or as a stable emulsion

(5) synthetic polynucleotides such as oligonucleotides containing CpG motif(s) (U.S. Pat. No. 6,207,646); and

(6) cytokines, such as interleukins (e.g., IL-1, IL-2, IL-4, IL-5, IL-6, IL-7, IL-12, IL-15, IL-18, etc.), interferons (e.g., gamma interferon), granulocyte macrophage colony stimulating factor (GM-CSF), macrophage colony stimulating factor (M-CSF), tumor necrosis factor (TNF), costimulatory molecules B7-1 and B7-2, etc.; and

(7) complement, such as a trimer of complement component C3d.

In another embodiment, the adjuvant is a mixture of 2, 3, or more of the above adjuvants, e.g., SBAS2 (an oil-in-water emulsion also containing 3-deacylated monophosphoryl lipid A and QS21).

Muramyl peptides include, but are not limited to, N-acetyl-muramyl-L-threonyl-D-isoglutamine (thr-MDP), N-acetyl-normuramyl-L-alanine-2-(1′-2′ dipalmitoyl-sn-glycero-3-hydroxyphosphoryloxy)-ethylamine (MTP-PE), etc.

In certain embodiments, the adjuvant is an aluminum salt. The aluminum salt adjuvant may be an alum-precipitated vaccine or an alum-adsorbed vaccine. Aluminum-salt adjuvants are well known in the art and are described, for example, in Harlow, E. and D. Lane (1988; Antibodies: A Laboratory Manual Cold Spring Harbor Laboratory) and Nicklas, W. (1992; Aluminum salts. Research in Immunology 143:489-493). The aluminum salt includes, but is not limited to, hydrated alumina, alumina hydrate, alumina trihydrate (ATH), aluminum hydrate, aluminum trihydrate, alhydrogel, Superfos, Amphogel, aluminum (III) hydroxide, aluminum hydroxyphosphate sulfate (Aluminum Phosphate Adjuvant (APA)), amorphous alumina, trihydrated alumina, or trihydroxyaluminum.

APA is an aqueous suspension of aluminum hydroxyphosphate. APA is manufactured by blending aluminum chloride and sodium phosphate in a 1:1 volumetric ratio to precipitate aluminum hydroxyphosphate. After the blending process, the material is size-reduced with a high-shear mixer to achieve a monodisperse particle size distribution. The product is then diafiltered against physiological saline and sterilized (either steam sterilization or autoclaving).

A commercially available Al(OH)₃ (e.g. Alhydrogel or Superfos of Denmark/Accurate Chemical and Scientific Co., Westbury, N.Y.) may be used to adsorb proteins. Adsorption of protein is dependent, in another embodiment, on the pI (Isoelectric pH) of the protein and the pH of the medium. A protein with a lower pI adsorbs to the positively charged aluminum ion more strongly than a protein with a higher pI. Aluminum salts may establish a depot of antigen that is released slowly over a period of 2-3 weeks, be involved in nonspecific activation of macrophages and complement activation, and/or stimulate innate immune mechanism (possibly through stimulation of uric acid). See, e.g., Lambrecht et al., 2009, Curr Opin Immunol 21:23.

Monovalent bulk aqueous conjugates are typically blended together and diluted to target 8 μg/mL for all serotypes except 6B, which will be diluted to target 16 μg/mL. Once diluted, the batch will be filter sterilized, and an equal volume of aluminum phosphate adjuvant added aseptically to target a final aluminum concentration of 250 μg/mL. The adjuvanted, formulated batch will be filled into single-use, 0.5 mL/dose vials.

In certain embodiments, the adjuvant is a CpG-containing nucleotide sequence, for example, a CpG-containing oligonucleotide, in particular, a CpG-containing oligodeoxynucleotide (CpG ODN). In another embodiment, the adjuvant is ODN 1826, which may be acquired from Coley Pharmaceutical Group.

“CpG-containing nucleotide,” “CpG-containing oligonucleotide,” “CpG oligonucleotide,” and similar terms refer to a nucleotide molecule of 6-50 nucleotides in length that contains an unmethylated CpG moiety. See, e.g., Wang et al., 2003, Vaccine 21:4297. In another embodiment, any other art-accepted definition of the terms is intended. CpG-containing oligonucleotides include modified oligonucleotides using any synthetic internucleoside linkages, modified base and/or modified sugar.

Methods for use of CpG oligonucleotides are well known in the art and are described, for example, in Sur et al., 1999, J Immunol. 162:6284-93; Verthelyi, 2006, Methods Mol Med. 127:139-58; and Yasuda et al., 2006, Crit Rev Ther Drug Carrier Syst. 23:89-110.

### Administration/Dosage

The compositions and formulations of the present invention can be used to protect or treat a human susceptible to infection, e.g., a pneumococcal infection, by means of administering the vaccine via a systemic or mucosal route. In one embodiment, the present invention provides a method of inducing an immune response to a S. pneumoniae capsular polysaccharide conjugate, comprising administering to a human an immunologically effective amount of an immunogenic composition of the present invention. In another embodiment, the present invention provides a method of vaccinating a human against a pneumococcal infection, comprising the step of administering to the human an immunologically effective amount of an immunogenic composition of the present invention.

Optimal amounts of components for a particular vaccine can be ascertained by standard studies involving observation of appropriate immune responses in subjects. For example, in another embodiment, the dosage for human vaccination is determined by extrapolation from animal studies to human data. In another embodiment, the dosage is determined empirically.

“Effective amount” of a composition of the invention refers to a dose required to elicit antibodies that significantly reduce the likelihood or severity of infectivity of a microbe, e.g., S. pneumonia, during a subsequent challenge.

The methods of the invention can be used for the prevention and/or reduction of primary clinical syndromes caused by microbes, e.g., S. pneumonia, including both invasive infections (meningitis, pneumonia, and bacteremia), and noninvasive infections (acute otitis media, and sinusitis).

Administration of the compositions of the invention can include one or more of: injection via the intramuscular, intraperitoneal, intradermal or subcutaneous routes; or via mucosal administration to the oral/alimentary, respiratory or genitourinary tracts. In one embodiment, intranasal administration is used for the treatment of pneumonia or otitis media (as nasopharyngeal carriage of pneumococci can be more effectively prevented, thus attenuating infection at its earliest stage).

The amount of conjugate in each vaccine dose may be selected as an amount that induces an immunoprotective response without significant, adverse effects. Such amount can vary depending upon the pneumococcal serotype. Generally, for polysaccharide-based conjugates, each dose will comprise 0.1 to 100 μg of each polysaccharide, particularly 0.1 to 10 μg, and more particularly 1 to 5 μg. For example, each dose can comprise 100, 150, 200, 250, 300, 400, 500, or 750 ng or 1, 1.5, 2, 3, 4, 5, 6, 7, 7.5, 8, 9, 10, 11, 12, 13, 14, 15, 16, 18, 20, 22, 25, 30, 40, 50, 60, 70, 80, 90, or 100 μg.

In one embodiment, the dose of the aluminum salt is 10, 15, 20, 25, 30, 50, 70, 100, 125, 150, 200, 300, 500, or 700 μg, or 1, 1.2, 1.5, 2, 3, 5 mg or more. In yet another embodiment, the dose of alum salt described above is per μg of recombinant protein.

According to any of the methods of the present invention and in one embodiment, the subject is human. In certain embodiments, the human patient is an infant (less than 1 year of age), toddler (approximately 12 to 24 months), or young child (approximately 2 to 5 years). In other embodiments, the human patient is an elderly patient (>65 years). The compositions of this invention are also suitable for use with older children, adolescents and adults (e.g., aged 18 to 45 years or 18 to 65 years).

In one embodiment of the methods of the present invention, a composition of the present invention is administered as a single inoculation. In another embodiment, the vaccine is administered twice, three times or four times or more, adequately spaced apart. For example, the composition may be administered at 1, 2, 3, 4, 5, or 6 month intervals or any combination thereof. The immunization schedule can follow that designated for pneumococcal vaccines. For example, the routine schedule for infants and toddlers against invasive disease caused by S. pneumoniae is 2, 4, 6 and 12-15 months of age. Thus, in a preferred embodiment, the composition is administered as a 4-dose series at 2, 4, 6, and 12-15 months of age.

The compositions of this invention may also include one or more proteins from S. pneumoniae. Examples of S. pneumoniae proteins suitable for inclusion include those identified in International Patent Application Publication Nos. WO 02/083855 and WO 02/053761.

### Formulations

The compositions of the invention can be administered to a subject by one or more method known to a person skilled in the art, such as parenterally, transmucosally, transdermally, intramuscularly, intravenously, intra-dermally, intra-nasally, subcutaneously, intra-peritoneally, and formulated accordingly.

In one embodiment, compositions of the present invention are administered via epidermal injection, intramuscular injection, intravenous, intra-arterial, subcutaneous injection, or intra-respiratory mucosal injection of a liquid preparation. Liquid formulations for injection include solutions and the like.

The composition of the invention can be formulated as single dose vials, multi-dose vials or as pre-filled syringes.

In another embodiment, compositions of the present invention are administered orally, and are thus formulated in a form suitable for oral administration, i.e., as a solid or a liquid preparation. Solid oral formulations include tablets, capsules, pills, granules, pellets and the like. Liquid oral formulations include solutions, suspensions, dispersions, emulsions, oils and the like.

Pharmaceutically acceptable carriers for liquid formulations are aqueous or nonaqueous solutions, suspensions, emulsions or oils. Examples of nonaqueous solvents are propylene glycol, polyethylene glycol, and injectable organic esters such as ethyl oleate. Aqueous carriers include water, alcoholic/aqueous solutions, emulsions or suspensions, including saline and buffered media. Examples of oils are those of animal, vegetable, or synthetic origin, for example, peanut oil, soybean oil, olive oil, sunflower oil, fish-liver oil, another marine oil, or a lipid from milk or eggs.

The pharmaceutical composition may be isotonic, hypotonic or hypertonic. However it is often preferred that a pharmaceutical composition for infusion or injection is essentially isotonic, when it is administrated. Hence, for storage the pharmaceutical composition may preferably be isotonic or hypertonic. If the pharmaceutical composition is hypertonic for storage, it may be diluted to become an isotonic solution prior to administration.

The isotonic agent may be an ionic isotonic agent such as a salt or a non-ionic isotonic agent such as a carbohydrate. Examples of ionic isotonic agents include but are not limited to sodium chloride (NaCl), calcium chloride (CaCl₂), potassium chloride (KCl) and magnesium chloride (MgCl₂). Examples of non-ionic isotonic agents include but are not limited to mannitol, sorbitol and glycerol.

It is also preferred that at least one pharmaceutically acceptable additive is a buffer. For some purposes, for example, when the pharmaceutical composition is meant for infusion or injection, it is often desirable that the composition comprises a buffer, which is capable of buffering a solution to a pH in the range of 4 to 10, such as 5 to 9, for example 6 to 8.

The buffer may for example be selected from the group consisting of TRIS, acetate, glutamate, lactate, maleate, tartrate, phosphate, citrate, carbonate, glycinate, histidine, glycine, succinate and triethanolamine buffer.

The buffer may furthermore for example be selected from USP compatible buffers for parenteral use, in particular, when the pharmaceutical formulation is for parenteral use. For example the buffer may be selected from the group consisting of monobasic acids such as acetic, benzoic, gluconic, glyceric and lactic; dibasic acids such as aconitic, adipic, ascorbic, carbonic, glutamic, malic, succinic and tartaric, polybasic acids such as citric and phosphoric; and bases such as ammonia, diethanolamine, glycine, triethanolamine, and TRIS.

Parenteral vehicles (for subcutaneous, intravenous, intraarterial, or intramuscular injection) include sodium chloride solution, Ringer's dextrose, dextrose and sodium chloride, lactated Ringer's and fixed oils. Intravenous vehicles include fluid and nutrient replenishers, electrolyte replenishers such as those based on Ringer's dextrose, and the like. Examples are sterile liquids such as water and oils, with or without the addition of a surfactant and other pharmaceutically acceptable adjuvants. In general, water, saline, aqueous dextrose and related sugar solutions, glycols such as propylene glycols or polyethylene glycol, are preferred liquid carriers, particularly for injectable solutions. Examples of oils are those of animal, vegetable, or synthetic origin, for example, peanut oil, soybean oil, olive oil, sunflower oil, fish-liver oil, another marine oil, or a lipid from milk or eggs.

The formulations of the invention may also contain a surfactant. Preferred surfactants include, but are not limited to: the polyoxyethylene sorbitan esters surfactants (commonly referred to as the Tweens); copolymers of ethylene oxide (EO), propylene oxide (PO), and/or butylene oxide (BO), sold under the DOWFAX™ tradename, such as linear EO/PO block copolymers; octoxynols, which can vary in the number of repeating ethoxy (oxy-1,2-ethanediyl) groups, with octoxynol-9 (Triton X-100, or t-octylphenoxypolyethoxyethanol) being of particular interest; (octylphenoxy)polyethoxyethanol (IGEPAL CA-630/NP-40); phospholipids such as phosphatidylcholine (lecithin); nonylphenol ethoxylates, such as the Tergitol™ NP series; polyoxyethylene fatty ethers derived from lauryl, cetyl, stearyl and oleyl alcohols (known as Brij surfactants), such as triethyleneglycol monolauryl ether (Brij 30); and sorbitan esters (commonly known as the SPANs), such as sorbitan trioleate (Span 85) and sorbitan monolaurate.

Preferred amounts of surfactants (% by weight) are: polyoxyethylene sorbitan esters (such as PS80) 0.01 to 1%, in particular about 0.1%; octyl- or nonylphenoxy polyoxyethanols (such as Triton X-100, or other detergents in the Triton series) 0.001 to 0.1%, in particular 0.005 to 0.02%; polyoxyethylene ethers (such as laureth 9) 0.1 to 20%, preferably 0.1 to 10% and in particular 0.1 to 1% or about 0.5%.

The formulation also contains a pH-buffered saline solution. The buffer may, for example, be selected from the group consisting of TRIS, acetate, glutamate, lactate, maleate, tartrate, phosphate, citrate, carbonate, glycinate, histidine, glycine, succinate, HEPES (4-(2-hydroxyethyl)-1-piperazineethanesulfonic acid), MOPS (3-(N-morpholino)propanesulfonic acid), MES (2-(N-morpholino)ethanesulfonic acid) and triethanolamine buffer. The buffer is capable of buffering a solution to a pH in the range of 4 to 10, 5.2 to 7.5, or 5.8 to 7.0. In certain aspect of the invention, the buffer selected from the group consisting of phosphate, succinate, histidine, MES, MOPS, HEPES, acetate or citrate. The buffer may furthermore, for example, be selected from USP compatible buffers for parenteral use, in particular, when the pharmaceutical formulation is for parenteral use. The concentrations of buffer will range from 1 mM to 50 mM or 5 mM to 50 mM. In certain aspects, the buffer is histidine at a final concentration of 5 mM to 50 mM, or succinate at a final concentration of 1 mM to 10 mM. In certain aspects, the histidine is at a final concentration of 20 mM±2 mM.

While the saline solution (i.e., a solution containing NaCl) is preferred, other salts suitable for formulation include but are not limited to, CaCl₂, KCl and MgCl₂ and combinations thereof. Non-ionic isotonic agents including but not limited to sucrose, trehalose, mannitol, sorbitol and glycerol may be used in lieu of a salt. Suitable salt ranges include, but not are limited to 25 mM to 500 mM or 40 mM to 170 mM. In one aspect, the saline is NaCl, optionally present at a concentration from 20 mM to 170 mM.

In a preferred embodiment, the formulations comprise a L-histidine buffer with sodium chloride.

In another embodiment, the pharmaceutical composition is delivered in a controlled release system. For example, the agent can be administered using intravenous infusion, a transdermal patch, liposomes, or other modes of administration. In another embodiment, polymeric materials are used; e.g. in microspheres or an implant.

The compositions of this invention may also include one or more proteins from S. pneumoniae. Examples of S. pneumoniae proteins suitable for inclusion include those identified in International Patent Application Publication Nos. WO 02/083855 and WO 02/053761.

### Analytical Methods

### Molecular Weight and Concentration Analysis of Conjugates Using HPSEC/UV/MALS/RI Assay

Conjugate samples are injected and separated by high performance size-exclusion chromatography (HPSEC). Detection is accomplished with ultraviolet (UV), multi-angle light scattering (MALS) and refractive index (RI) detectors in series. Protein concentration is calculated from UV280 using an extinction coefficient. Polysaccharide concentration is deconvoluted from the RI signal (contributed by both protein and polysaccharide) using the do/dc factors which are the change in a solution's refractive index with a change in the solute concentration reported in mL/g. Average molecular weight of the samples are calculated by Astra software (Wyatt Technology Corporation, Santa Barbara, Calif.) using the measured concentration and light scattering information across the entire sample peak. There are multiple forms of average values of molecular weight for polydispersed molecules. For example, number-average molecular weight Mn, weight-average molecular weight Mw, and z-average molecular weight Mz (Molecules, 2015, 20:10313-10341). Unless specified, the term “molecular weight”, as used throughout the specification, is the weight-average molecular weight.

### Determination of Lysine Consumption in Conjugated Protein as a Measure of the Number of Covalent Attachments Between Polysaccharide and Carrier Protein

The Waters AccQ-Tag amino acid analysis (AAA) is used to measure the extent of conjugation in conjugate samples. Samples are hydrolyzed using vapor phase acid hydrolysis in the Eldex workstation, to break the carrier proteins down into their component amino acids. The free amino acids are derivatized using 6-aminoquinolyl-N-hydroxysuccinimidyl carbamate (AQC). The derivatized samples are then analyzed using UPLC with UV detection on a C18 column. The average protein concentration is obtained using representative amino acids other than lysine. Lysine consumption during conjugation (i.e., lysine loss) is determined by the difference between the average measured amount of lysine in the conjugate and the expected amount of lysine in the starting protein.

### Free Polysaccharide Testing

Free polysaccharide (i.e., polysaccharide that is not conjugated with CRM197) in the conjugate sample is measured by first precipitating free protein and conjugates with deoxycholate (DOC) and hydrochloric acid. Precipitates are then filtered out and the filtrates are analyzed for free polysaccharide concentration by HPSEC/UV/MALS/RI. Free polysaccharide is calculated as a percentage of total polysaccharide measured by HPSEC/UV/MALS/RI.

### Free Protein Testing

Free polysaccharide, polysaccharide-CRM197 conjugate, and free CRM197 in the conjugate samples are separated by capillary electrophoresis in micellar electrokinetic chromatography (MEKC) mode. Briefly, samples are mixed with MEKC running buffer containing 25 mM borate, 100 mM SDS, pH 9.3, and are separated in a preconditioned bare-fused silica capillary. Separation is monitored at 200 nm and free CRM197 is quantified with a CRM197 standard curve. Free protein results are reported as a percentage of total protein content determined by the HPSEC/UV/MALS/RI procedure.

### Polysaccharide Degree of Activation Assay

Conjugation occurs through reductive amination between the activated aldehydes and mainly lysine residues on the carrier protein. The level of activation, as mole of aldehyde per mole of polysaccharide repeat unit, is important to control the conjugation reactions.

In this assay, polysaccharide is derivatized with 2.5 mg/mL thiosemicarbazide (TSC) at pH4.0 to introduce a chromophore (derivatization of activated polysaccharide for serotype 1, 5, 9V uses 1.25 mg/mL TSC). The derivatization reaction was allowed to proceed to reach a plateau. The actual time varies depending on reaction speed of each serotype. TSC-Ps is then separated from TSC and other low molecular weight components by high performance size exclusion chromatography. The signal is detected by UV absorbance at 266 nm. The level of activated aldehyde is calculated either against standard curve injections of Mono-TSC or directly using predetermined extinction coefficient. Mono-TSC is a synthesized thiosemicarbazone derivative of monosaccharide. The aldehyde level is then converted as moles of aldehyde per mole of repeat unit (Ald/RU) using the Ps concentration measured by HPSEC/UV/MALS/RI assay.

Having described various embodiments of the invention with reference to the accompanying description, it is to be understood that the invention is not limited to those precise embodiments, and that various changes and modifications may be effected therein by one skilled in the art without departing from the scope or spirit of the invention as defined in the appended claims.

The following examples illustrate, but do not limit the invention.

### Example 1

Preparation of S. pneumoniae 35B Capsular Polysaccharide

### Fermentation

Methods of culturing pneumococci are well known in the art. See, e.g., Chase, 1967, Methods of Immunology and Immunochemistry 1:52. Methods of preparing pneumococcal capsular polysaccharides are also well known in the art. See, e.g., European Patent No. EP 0 497 524 B1. The process described below generally follows the method described in European Patent No. EP 0 497 524 B1 and is generally applicable to all pneumococcal serotypes except where specifically modified.

Isolates of pneumococcal subtype 35B were obtained from the Merck Culture Collection. Where needed, subtypes can be differentiated on the basis of Quelling reaction using specific antisera. See, e.g., U.S. Pat. No. 5,847,112. The obtained isolates were further clonally isolated by plating serially in two stages on agar plates consisting of an animal-component free medium containing soy peptone, yeast extract, and glucose without hemin. Clonal isolates for each serotype were further expanded in liquid culture using animal-component free media containing soy peptone, yeast extract, HEPES, sodium chloride, sodium bicarbonate, potassium phosphate, glucose, and glycerol to prepare the pre-master cell banks.

The production of S. pneumoniae serotype 35B consisted of a cell expansion and batch production fermentation followed by chemical inactivation prior to downstream purification. A thawed cell bank vial was expanded using a shake flask or culture bottle containing a pre-sterilized animal-component free growth media containing soy peptone or soy peptone ultrafiltrate, yeast extract or yeast extract ultrafiltrate, HEPES, sodium chloride, sodium bicarbonate, potassium phosphate, and glucose. The cell expansion culture was grown in a sealed shake flask or bottle to minimize gas exchange with temperature and agitation control. After achieving a specified culture density, as measured by optical density at 600 nm, a portion of the cell expansion culture was transferred to a production fermentor containing pre-sterilized animal-component free growth media containing soy peptone or soy peptone ultrafiltrate, yeast extract or yeast extract ultrafiltrate, sodium chloride, potassium phosphate, and glucose. Temperature, pH, pressure, and agitation were controlled. Airflow overlay was also controlled as sparging was not used.

The batch fermentation was terminated via the addition of a chemical inactivating agent, phenol, when glucose was nearly exhausted. Pure phenol was added to a final concentration of 0.8-1.2% to inactivate the cells and liberate the capsular polysaccharide from the cell wall. Primary inactivation occurs for a specified time within the fermentor where temperature and agitation continue to be controlled. After primary inactivation, the batch was transferred to another vessel where it was held for an additional specified time at controlled temperature and agitation for complete inactivation. This was confirmed by either microbial plating techniques or by verification of the phenol concentration and specified time. The inactivated broth was then purified.

### Purification of Ps

The purification of the pneumococcal polysaccharide consisted of several centrifugation, depth filtration, concentration/diafiltration operations, and precipitation steps. All procedures were performed at room temperature unless otherwise specified.

Inactivated broth from the fermentor cultures of S. pneumoniae were flocculated with a cationic polymer (such as BPA-1000, Petrolite “Tretolite” and “Spectrum 8160” and poly(ethyleneimine), “Millipore pDADMAC”). The cationic polymers binded to the impurity protein, nucleic acids and cell debris. Following the flocculation step and an aging period, flocculated solids were removed via centrifugation and multiple depth filtration steps. Clarified broth was concentrated and diafiltered using a 100 kDa to 500 kDa MWCO (molecular weight cutoff) filter. Diafiltration was accomplished using Tris, MgCl₂ buffer and sodium phosphate buffer. Diafiltration removed residual nucleic acid and protein.

Further, impurities removal was accomplished by reprecipitation of the polysaccharide in sodium acetate and phenol with denatured alcohol and/or isopropanol. During the phenol precipitation step, sodium acetate in sodium phosphate saline buffer and phenol (liquefied phenols or solid phenols) was charged to the diafiltered retentate. Alcohol fractionation of the polysaccharide was then conducted in two stages. In the first stage a low percent alcohol was added to the preparation to precipitate cellular debris and other unwanted impurities, while the crude polysaccharide remained in solution. The impurities were removed via a depth filtration step. The polysaccharide was then recovered from the solution by adding additional isopropanol or denatured alcohol to the batch. The precipitated polysaccharide pellet was recovered by centrifugation, triturated and dried as a powder and stored frozen at −70° C.

### Example 2

Activation of S. pneumoniae Serotype 35B Polysaccharide

Purified pneumococcal capsular Ps powder was dissolved in water and 0.45-micron filtered. Dissolved polysaccharide was homogenized to reduce Ps solution viscosity. Homogenization pressure and number of passes through the homogenizer were controlled to 100 bar/5 passes. Homogenized polysaccharide was concentrated and diafiltered against water using a 5 kDa NMWCO tangential flow ultrafiltration membrane.

The polysaccharide solution was adjusted to 22° C. and pH 5 with a sodium acetate buffer. Polysaccharide activation was initiated with the addition of a 100 mM sodium metaperiodate solution. The amount of sodium metaperiodate added was 0.01, 0.03, 0.05, 0.07, 0.09 or 0.11 moles of sodium metaperiodate per mole of polysaccharide repeating unit to achieve a target level of polysaccharide activation (moles aldehyde per mole of polysaccharide repeating unit). The oxidation reaction proceeded for 1 hour at 22° C. Activated polysaccharides were dialyzed at approximately 4° C. against 10 nil potassium phosphate, pH 6.4 followed by distilled water for a total of 3 days using a 5 kDa NMWCO dialysis cassette.

As demonstrated in Table 1, when sodium metaperiodate is added to the activation reaction, the S. pneumoniae serotype 35B polysaccharide chain undergoes a size reduction and a simultaneous increase in the number of aldehydes per repeat unit. As the sodium metaperiodate molar equivalents (charged to the reaction) are increased, the size of the 35B polysaccharide is reduced and the number of reactive aldehydes per repeat unit are increased, suggesting that activation is cleaving the polysaccharide main chain at the acrylic triol sites. Table 1. Attributes of S. pneumoniae Serotype 35B Polysaccharide at Different Target Activation Levels

| | Metaperiodate | | Oxidized Ps |
|---|---|---|---|
| | Charge (molar | Oxidized Ps | Activation Level |
| Arm | equivalents) | Mw (kDa) | (Aldehydes/Repeat Unit) |
| 1 | 0.01 | 160 | 0.012 |
| 2 | 0.03 | 88 | 0.035 |
| 3 | 0.05 | 66 | 0.051 |
| 4 | 0.07 | 55 | 0.072 |
| 5 | 0.09 | 47 | 0.079 |
| 6 | 0.11 | 42 | 0.095 |

### Example 3

Conjugation of S. pneumoniae Serotype 35B Polysaccharide to CRM197

### Polysaccharide Activation

Polysaccharides were activated and purified as described in Example 2.

Polysaccharide conjugation to CRM197

Purified CRM197, obtained through expression in Pseudomonas fluorescens as previously described (WO 2012/173876 A1), was diafiltered against 2 mM phosphate, pH 7.2 buffer using a 5 kDa NMWCO tangential flow ultrafiltration membrane and 0.2-micron filtered.

Activated polysaccharides were formulated for lyophilization at 6 mg Ps/mL with sucrose concentration of 5% w/v. CRM197 was formulated for lyophilization at 6 mg Pr/mL with sucrose concentration of 1% w/v.

Formulated Ps and CRM197 solutions were individually lyophilized. Lyophilized Ps and CRM197 materials were redissolved individually in equal volumes of DMSO. Arms with salt had sodium chloride spiked into the dissolved Ps to a concentration of 10 mM sodium chloride. The polysaccharide and CRM197 solutions were blended to achieve a polysaccharide concentration of 6 g Ps/L (Arms 1-6) or 7.5 g Ps/L (Arms 7-12) and a polysaccharide to CRM197 mass ratio of 3. Conjugation proceeded for 3 hours at 34° C.

Reduction with Sodium Borohydride

Sodium borohydride (2 moles per mole of polysaccharide repeating unit) was added following the conjugation reaction and incubated for 1 hour at 34° C. The batch was diluted into 150 mM sodium chloride, with approximately 0.025% (w/v) polysorbate 20, at approximately 4° C. Potassium phosphate buffer was then added to neutralize the pH. The batch was dialyzed at approximately 4° C. for 3 days against 150 mM sodium chloride, 0.05% (w/v) polysorbate 20, using a 300 kDa NMWCO dialysis cassette.

As demonstrated in Table 2, the number of reactive aldehydes per repeat unit and the size of the S. pneumoniae serotype 35B polysaccharide chain (which are controlled by the amount of periodate charged during the activation step) directly influence the 35B conjugate attributes. As the size of the oxidized 35B polysaccharide increases, the number of reactive aldehydes per repeat unit decreases. Therefore, oxidized 35B polysaccharide molecules with lower aldehydes per repeat unit result in 35B conjugates with increased size but (1) decreased lysine consumption, (2) increased free polysaccharide percentage and (3) increased free protein percentage. On the other hand, oxidized 35B polysaccharide molecules with an increased number of aldehydes per repeat unit possess smaller polysaccharide sizes, resulting in 35B conjugates with sizes below 1000 kD.

Note that dialysis is less effective at clearing free protein and free polysaccharide compared to full scale purification methods, such as ultrafiltration (see Table 8 for examples of conjugates purified using ultrafiltration). The free polysaccharide and free protein values in Table 2 are used to demonstrate the trends in conjugation efficiency with regards to polysaccharide activation level.

TABLE 2
Attributes of S. pneumoniae Serotype 35B Polysacharride-Protein Conjugates

| | | Oxidized Ps | | | | | Free |
|---|---|---|---|---|---|---|---|
| | | Activation | | | Lysine | | Protein/ |
| | Oxidized Ps | Level | Conjugate | | Consumption | Free Ps/ | Total |
| | Mn/Mw | (Aldehydes/ | Mn/Mw | | (mol/mol | Total Ps | Protein |
| Arm | (kDa) | Repeat Unit) | (kD) | Ps:Pr | CRM197) | (%) | (%) |
| 1 | 113/160 | 0.012 | 1782/3352 | 2.86 | 2.8 | 63 | 44 |
| 2 | 59/88 | 0.035 | 424/1395 | 2.61 | 4.5 | 44 | 14 |
| 3 | 42/66 | 0.051 | 284/860 | 2.54 | 5.8 | 39 | 6 |
| 4 | 35/55 | 0.072 | 250/639 | 2.42 | 7.1 | 37 | 3 |
| 5 | 26/47 | 0.079 | 312/595 | 2.09 | 7.7 | 30 | <2 |
| 6 | 23/42 | 0.095 | 179/452 | 2.38 | 9.0 | 36 | <2 |
| 7 | 113/160 | 0.012 | 2346/3651 | 3.51 | 3.0 | 69 | 54 |
| 8 | 59/88 | 0.035 | 1284/2498 | 2.60 | 4.6 | 49 | 15 |
| 9 | 42/66 | 0.051 | 1019/2022 | 2.21 | 5.8 | 39 | 5 |
| 10 | 35/55 | 0.072 | 503/1272 | 2.08 | 6.7 | 32 | 3 |
| 11 | 26/47 | 0.079 | 416/1017 | 2.06 | 7.3 | 32 | 1 |
| 12 | 23/42 | 0.095 | 383/840 | 1.92 | 8.4 | 25 | <1 |

### Example 4

Effect of Temperature on S. pneumoniae Serotype 35B Polysaccharide-Protein Conjugate Attributes

### Polysaccharide Activation

Purified pneumococcal serotype 35B capsular Ps powder was dissolved in water and 0.45-micron filtered. Dissolved polysaccharide was concentrated and diafiltered against water using a 5 kDa NMWCO tangential flow ultrafiltration membrane.

The polysaccharide solution was then adjusted to 22° C. and pH 5 with a sodium acetate buffer. Polysaccharide activation was initiated with the addition of a 10 mM sodium metaperiodate solution. The amount of sodium metaperiodate added was 0.047 moles of sodium metaperiodate per mole of polysaccharide repeating unit to achieve a target level of polysaccharide activation (moles aldehyde per mole of polysaccharide repeating unit). The oxidation reaction proceeded for 1 hour at 22° C.

The activated product was diafiltered against 10 mM potassium phosphate, pH 6.4 followed by diafiltration against water using a 5 kDa NMWCO tangential flow ultrafiltration membrane. Ultrafiltration was conducted at 2-8° C.

### Polysaccharide Conjugation to CRM197

Purified CRM197, obtained through expression in Pseudomonas fluorescens as previously described (WO 2012/173876 A1), was diafiltered against 2 mM phosphate, pH 7.2 buffer using a 5 kDa NMWCO tangential flow ultrafiltration membrane and 0.2-micron filtered.

Activated polysaccharides were formulated for lyophilization at 6 mg Ps/mL with sucrose concentration of 5% w/v and sodium chloride concentration of 10 mM. CRM197 was formulated for lyophilization at 6 mg Pr/mL with sucrose concentration of 1% w/v.

Formulated Ps and CRM197 solutions were individually lyophilized. Lyophilized Ps and CRM197 materials were redissolved individually in equal volumes of DMSO. The polysaccharide and CRM197 solutions were blended to achieve a polysaccharide concentration of 6 g Ps/L and a polysaccharide to CRM197 mass ratio of 3. Sodium cyanoborohydride (1 mole per mole of polysaccharide repeating unit) was added, and the conjugation reaction proceeded for 3 hours at 28° C., 30° C., 34° C. or 38° C.

Reduction with Sodium Borohydride

Sodium borohydride (2 moles per mole of polysaccharide repeating unit) was added following the conjugation reaction and incubated for 1 hour at the same temperature at conjugation. The batch was diluted into 150 mM sodium chloride, with approximately 0.025% (w/v) polysorbate 20, at approximately 4° C. Potassium phosphate buffer was then added to neutralize the pH. The batch was dialyzed at 2-8° C. for 3 days against 10 mM histidine in 150 mM sodium chloride, pH 7.0, with 0.015% (w/v) polysorbate 20 using a 300 kDa MWCO dialysis cassette.

As demonstrated in Table 3, the temperature implemented during the conjugation reaction has an effect on S. pneumoniae 35B polysaccharide/CRM197 conjugate attributes. As the conjugation temperature increases from 22° C. to 38° C., the 35B conjugates experience a decrease in free polysaccharide fraction, suggesting that conjugation is more effective at higher temperatures in this range.

TABLE 3
Effect on Conjugation Temperature on Attributes of S. pneumoniae
Serotype 35B Polysacharride-Protein Conjugates

| | | | | Lysine | | |
|---|---|---|---|---|---|---|
| | | Conjugate | | Consumption | Free Ps/ | Free Protein/ |
| | Temp | Mn/Mw | | (mol/mol | Total Ps | Total Protein |
| Arm | (° C.) | (kD) | Ps:Pr | CRM197) | (%) | (%) |
| 1 | 22 | 695/1487 | 2.09 | 5.6 | 45.0 | 10.8 |
| 2 | 30 | 663/1449 | 2.26 | 6.2 | 38.2 | 8.9 |
| 3 | 34 | 809/1602 | 1.99 | 6.3 | 27.9 | 9.7 |
| 4 | 38 | 831/1577 | 1.92 | 6.5 | 22.3 | 9.7 |

### Example 5

Effect of Water Concentration on S. pneumoniae Serotype 35B Polysaccharide-Protein

### Conjugate Attributes

Conjugation of S. pneumoniae serotype 35B polysaccharide to CRM197 is performed in an organic solvent such as DMSO. Even in an organic background, water can be introduced into the conjugation reaction as other components are added, e.g. spiking in a reducing agent, or over time as DMSO is hygroscopic. The effect of water content on S. pneumoniae serotype 35B polysaccharide-protein (CRM197) conjugate attributes was studied by spiking in distilled water at the start of conjugation.

### Polysaccharide Activation

Purified pneumococcal serotype 35B capsular Ps powder was dissolved in water and 0.45-micron filtered. Filtered dissolved polysaccharide was concentrated and diafiltered against water using a 5 kDa NMWCO tangential flow ultrafiltration membrane.

The polysaccharide solution was then adjusted to 22° C. and pH 5 with a sodium acetate buffer. Polysaccharide activation was initiated with the addition of a 10 mM sodium metaperiodate solution. The amount of sodium metaperiodate added was 0.047 moles of sodium metaperiodate per mole of polysaccharide repeating unit to achieve a target level of polysaccharide activation (moles aldehyde per mole of polysaccharide repeating unit). The oxidation reaction proceeded for 2 hours at 22° C.

The activated product was diafiltered against 10 mM potassium phosphate, pH 6.4 followed by diafiltration against water using a 5 kDa NMWCO tangential flow ultrafiltration membrane. Ultrafiltration was conducted at 2-8° C.

### Polysaccharide Conjugation to CRM197

Purified CRM197, obtained through expression in Pseudomonas fluorescens as previously described (WO 2012/173876 A1), was diafiltered against 2 mM phosphate, pH 7.2 buffer using a 5 kDa NMWCO tangential flow ultrafiltration membrane and 0.2-micron filtered.

Activated polysaccharides were formulated for lyophilization at 6 mg Ps/mL with sucrose concentration of 5% w/v. CRM197 was formulated for lyophilization at 6 mg Pr/mL with sucrose concentration of 1% w/v.

Formulated Ps and CRM197 solutions were individually lyophilized. Lyophilized Ps and CRM197 materials were redissolved individually in equal volumes of DMSO. The polysaccharide and CRM197 solutions were blended to achieve a polysaccharide concentration of 6 g Ps/L and a polysaccharide to CRM197 mass ratio of 3. Water was immediately spiked into the conjugation reaction to a target percentage. The mass ratio was selected to control the polysaccharide to CRM197 ratio in the resulting conjugate. The conjugation reaction proceeded for 3 hours at 34° C.

Reduction with Sodium Borohydride

Sodium borohydride (2 moles per mole of polysaccharide repeating unit) was added following the conjugation reaction and incubated for 1 hour at 34° C. The batch was diluted into 150 mM sodium chloride, with approximately 0.025% (w/v) polysorbate 20, at approximately 4° C. Potassium phosphate buffer was then added to neutralize the pH. The batch was dialyzed at 2-8° C. for 3 days against 10 mM histidine in 150 mM sodium chloride, pH 7.0, with 0.015% (w/v) polysorbate 20 using a 300 kDa MWCO dialysis cassette.

As demonstrated in Table 4, free polysaccharide and free protein increase with increasing percent water content in the conjugation reaction. This is likely due to protein aggregating at the higher concentrations of water, therefore for effective conjugation care must be taken to minimize water content during the conjugation reaction.

TABLE 4
Effect of Water Content During Conjugation on Attributes of S. pneumoniae
Serotype 35B Polysacharride-Protein (CRM197) Conjugates

| | | | | Lysine | | |
|---|---|---|---|---|---|---|
| | Water Content in | Conjugate | | Consumption | Free Ps/ | Free Protein/ |
| | the Conjugation | Mn/Mw | | (mol/mol | Total Ps | Total Protein |
| Arm | Reaction (%) | (kD) | Ps:Pr | CRM197) | (%) | (%) |
| 1 | 0 | 845/1747 | 2.41 | 5.4 | 30 | 8 |
| 2 | 0.10 | 671/1302 | 2.40 | 8.9 | 32 | 8 |
| 3 | 0.20 | 556/1117 | 2.38 | 6.0 | 43 | 11 |
| 4 | 0.30 | 524/1123 | 2.32 | — | 47 | 12 |
| 5 | 0.40 | 569/1143 | 2.35 | 6.6 | 46 | 12 |
| 6 | 0.60 | 528/1249 | 2.40 | 5.3 | 59 | >15 |
| 7 | 0.80 | 593/1466 | 2.17 | 4.9 | 60 | >15 |
| 8 | 1.00 | 977/2106 | 1.68 | 4.8 | 61 | >15 |
| 9 | 1.20 | 821/2192 | 1.94 | 4.5 | 75 | >15 |

### Example 6

Preparation of S. pneumoniae Serotype 35B Polysaccharide-Protein Conjugate with Sodium Chloride in the Lyophilization Formulation

Salts may be spiked into the conjugation reaction to improve conjugate attributes, but it also introduces water into the conjugation reaction. Water interferes with the serotype 35B polysaccharide conjugation reaction by promoting protein and polysaccharide aggregation as shown in Example 5 above. The water content in the conjugation reaction was minimized by incorporating sodium chloride in the pre-lyophilization formulation. The reduction in water content during the conjugation reaction resulted in conjugates with increased size, increased lysine loss and decreased free protein and free polysaccharide.

### Polysaccharide Activation

Purified pneumococcal serotype 35B capsular Ps powder was dissolved in water and 0.45-micron filtered. Filtered dissolved polysaccharide was concentrated and diafiltered against water using a 5 kDa NMWCO tangential flow ultrafiltration membrane.

The polysaccharide solution was then adjusted to 22° C. and pH 5 with a sodium acetate buffer. Polysaccharide activation was initiated with the addition of a 10 mM sodium metaperiodate solution. The amount of sodium metaperiodate added was 0.047 moles of sodium metaperiodate per mole of polysaccharide repeating unit to achieve a target level of polysaccharide activation (moles aldehyde per mole of polysaccharide repeating unit). The oxidation reaction proceeded for 2 hours at 22° C.

The activated product was diafiltered against 10 mM potassium phosphate, pH 6.4 followed by diafiltration against water using a 5 kDa NMWCO tangential flow ultrafiltration membrane. Ultrafiltration was conducted at 2-8° C.

### Polysaccharide Conjugation to CRM197

Purified CRM197, obtained through expression in Pseudomonas fluorescens as previously described (WO 2012/173876 A1), was diafiltered against 2 mM phosphate, pH 7.2 buffer using a 5 kDa NMWCO tangential flow ultrafiltration membrane and 0.2-micron filtered.

Activated polysaccharides were formulated for lyophilization at 6 mg Ps/mL with sucrose concentration of 5% w/v. Different levels of sodium chloride were spiked into the Ps-PreLyo for certain arms as described in Table 5. CRM197 was formulated for lyophilization at 6 mg Pr/mL with sucrose concentration of 1% w/v.

Formulated Ps and CRM197 solutions were individually lyophilized. Lyophilized Ps and CRM197 materials were redissolved individually in equal volumes of DMSO. The polysaccharide and CRM197 solutions were blended to achieve a polysaccharide concentration of 6 g Ps/L, a polysaccharide to CRM197 mass ratio of 3 and a sodium chloride concentration of 0 mM, 5 mM, 10 mM or 15 mM. As listed in Table 5, the addition of sodium chloride was achieved either through Ps pre-lyo or spiked as an aqueous solution into the Ps-DMSO after Ps dissolution. The conjugation reaction proceeded for 3 hours at 34° C.

Reduction with Sodium Borohydride

Sodium borohydride (2 moles per mole of polysaccharide repeating unit) was added following the conjugation reaction and incubated for 1 hour at 34° C. The batch was diluted into 150 mM sodium chloride, with approximately 0.025% (w/v) polysorbate 20, at approximately 4° C. Potassium phosphate buffer was then added to neutralize the pH. The batch was dialyzed at 2-8° C. for 3 days against 10 mM histidine in 150 mM sodium chloride, pH 7.0, with 0.015% (w/v) polysorbate 20 using a 300 kDa MWCO dialysis cassette.

As demonstrated in Table 5, spiking aqueous sodium chloride into the conjugation reaction (Arms 2-4) yields conjugate attributes similar to conjugates without sodium chloride (Arm 1). Conversely, adding sodium chloride to the reaction before lyophilization (Arms 5-7) yields conjugates with increased Mw and decreased free Ps relative to the no sodium chloride condition.

TABLE 5
Effect of Sodium Chloride During Conjugation on Attributes of
S. pneumoniae Serotype 35B Polysacharride-Protein Conjugates

| A | B | C | D | E | F | G | H | I |
|---|---|---|---|---|---|---|---|---|
| 1 | N/A | 0 | 0 | 485/981 | 2.45 | 6.9 | 31 | 7 |
| 2 | Ps | 0.10 | 5 | 595/1130 | 2.29 | 7.2 | 30 | 6 |
| | Dissolution | | | | | | | |
| 3 | Ps | 0.20 | 10 | 627/1219 | 2.22 | 6.4 | 32 | 7 |
| | Dissolution | | | | | | | |
| 4 | Ps | 0.30 | 15 | 669/1277 | 2.23 | 7.0 | 30 | 6 |
| | Dissolution | | | | | | | |
| 5 | Ps Pre-Lyo | 0 | 5 | 799/1613 | 2.44 | 7.6 | 25 | 6 |
| 6 | Ps Pre-Lyo | 0 | 10 | 896/1676 | 2.42 | 7.7 | 22 | 5 |
| 7 | Ps Pre-Lyo | 0 | 15 | 883/1598 | 2.42 | 7.6 | 20 | 4 |

A) Arm;
B) Step Sodium Chloride Added;
C) Water Content in the Conjugation Reaction (%);
D) Sodium Chloride in the Congugation Reaction (mM);
E) Conjugate Mn/Mw (kD);
F) Ps:Pr;
G) Lysine Consumption (mol/mol CRM197);
H) Free Ps/Total Ps (%);
I) Free Protein/Total Protein (%).

### Example 7

Preparation of S. pneumoniae Serotype 35B Polysaccharide-Protein Conjugates for Mouse Study

As described below, S. pneumoniae serotype 35B polysaccharide was dissolved, chemically activated and buffer-exchanged by ultrafiltration. Activated polysaccharide and purified CRM197 were individually lyophilized and redissolved in DMSO. Redissolved polysaccharide and CRM197 solutions were then combined and conjugated. The resulting conjugate was purified by ultrafiltration prior to a final 0.2-micron filtration. Several process parameters within each step, such as pH, temperature, concentration, and time were controlled to yield conjugates with desired attributes.

### Polysaccharide Activation

Purified pneumococcal serotype 35B capsular Ps powder was dissolved in water and 0.45-micron filtered. If applicable, dissolved polysaccharide was homogenized to reduce Ps solution viscosity. Homogenization pressure and number of passes through the homogenizer were controlled to reduce polysaccharide viscosity. Polysaccharide was concentrated and diafiltered against water using a 5 kDa NMWCO tangential flow ultrafiltration membrane.

The polysaccharide solution was then adjusted to 22° C. and pH 5 with a sodium acetate buffer. Polysaccharide activation was initiated with the addition of a 10 mM sodium metaperiodate solution. The amount of sodium metaperiodate added was 0.038 or 0.047 moles of sodium metaperiodate per mole of polysaccharide repeating unit to achieve a target level of polysaccharide activation (moles aldehyde per mole of polysaccharide repeating unit). The oxidation reaction proceeded for 1-2 hours at 22° C.

The activated product was diafiltered against 10 mM potassium phosphate, pH 6.4 followed by diafiltration against water using a 5 kDa NMWCO tangential flow ultrafiltration membrane. Ultrafiltration was conducted at 2-8° C.

### Polysaccharide Conjugation to CRM197

Purified CRM197, obtained through expression in Pseudomonas fluorescens as previously described (WO 2012/173876 A1), was diafiltered against 2 mM phosphate, pH 7.2 buffer using a 5 kDa NMWCO tangential flow ultrafiltration membrane and 0.2-micron filtered.

Activated polysaccharides were formulated for lyophilization at 6 mg Ps/mL with sucrose concentration of 5% w/v and sodium chloride for Group 6. CRM197 was formulated for lyophilization at 6 mg Pr/mL with sucrose concentration of 1% w/v.

Formulated Ps and CRM197 solutions were individually lyophilized. Lyophilized Ps and CRM197 materials were redissolved individually in equal volumes of DMSO. Groups 3, 4 and 8 had sodium chloride spiked into the Ps-DMSO solution. The polysaccharide and CRM197 solutions were blended to achieve a polysaccharide concentration of 6 g Ps/L and a polysaccharide to CRM197 mass charge ratio of 1.5, 2.2 or 3.0. The conjugation reaction proceeded for 3 to 6 hours at 34° C. Conjugation parameters are summarized in Table 6.

Reduction with Sodium Borohydride

Sodium borohydride (2 moles per mole of polysaccharide repeating unit) was added following the conjugation reaction and incubated for 1 hour at 34° C. The batch was diluted into 150 mM sodium chloride, with approximately 0.025% (w/v) polysorbate 20, at approximately 4° C. Potassium phosphate buffer was then added to neutralize the pH. The batch was concentrated and diafiltered at 2-8° C. against 10 mM histidine in 150 mM sodium chloride, pH 7.0, with 0.015% (w/v) polysorbate 20 using a 300 kDa NMWCO tangential flow ultrafiltration membrane.

### Final Filtration and Product Storage

The retentate batch was 0.5/0.2 micron filtered then diluted with additional 10 mM histidine in 150 mM sodium chloride, pH 7.0 with 0.015% (w/v) polysorbate 20, dispensed into aliquots and frozen at ≤−60° C. The resulting conjugate attributes are summarized in Table 7.

TABLE 6
S. pneumoniae Serotype 35B Polysacharride-
Protein Conjugation Parameters for Mouse Study

| | Periodate | Charged | Sodium Chloride | |
|---|---|---|---|---|
| | Charge | Mass | Concentration in | |
| | (molar | Ps:CRM197 | Conjugation | Conjugation |
| Group | equivalents) | (g:g) | Reaction (mM) | Time (hr) |
| 1, 7 | 0.047 | 3.0 | 0 | 4 |
| 2 | 0.047 | 2.2 | 0 | 6 |
| 3 | 0.047 | 1.5 | 5 | 3 |
| 4, 8 | 0.047 | 1.5 | 5 | 6 |
| 5 | 0.038 | 3.0 | 0 | 3 |
| 6 | 0.038 | 3.0 | 5 | 3 |

TABLE 7
Attributes of S. pneumoniae Serotype 35B Polysacharride-
Protein Conjugates for Mouse Study

| | | Oxidized Ps | | | | | Free |
|---|---|---|---|---|---|---|---|
| | | Activation | | | Lysine | | Protein/ |
| | Oxidized Ps | Level | Conjugate | | Consumption | Free Ps/ | Total |
| | Mn/Mw | (Aldehydes/ | Mn/Mw | | (mol/mol | Total Ps | Protein |
| Group | (kDa) | Repeat Unit) | (kD) | Ps:Pr | CRM197) | (%) | (%) |
| 1, 7 | 54/74 | 0.046 | 650/1081 | 1.8 | 7.3 | 10% | 5% |
| 2 | 55/71 | 0.045 | 1070/2014 | 1.4 | 5.3 | 7% | 8% |
| 3 | 54/70 | 0.049 | 1440/2823 | 0.9 | 5.1 | 5% | 10% |
| 4, 8 | 50/69 | 0.046 | 2762/4903 | 0.8 | 4.3 | 7% | 17% |
| 5 | 57/82 | 0.040 | 1030/1849 | 1.7 | 6.2 | 15% | 11% |
| 6 | 57/82 | 0.040 | 1256/1974 | 1.7 | 6.3 | 11% | 10% |

### Example 8

### Formulation of Pneumococcal Conjugate Vaccines for Mouse Study

Individual 35B-CRM197 conjugates, prepared utilizing different processes as described in the Examples above, were used for the formulation of monovalent pneumococcal conjugate vaccines.

The monovalent drug product is prepared using Pneumococcal polysaccharide 35B-CRM197 conjugate and is formulated in 20 mM histidine pH 5.8 and 150 mM sodium chloride and 0.1% w/v Polysorbate-20 (PS-20) at targeted 4.0 μg/mL Pneumococcal polysaccharide antigen. For groups 7 and 8 in the mouse study, the formulation is prepared with 250 μg [Al]/mL in the form of Aluminum Phosphate as the adjuvant.

The required volume of bulk conjugate needed to obtain the target concentration of individual Pneumococcal polysaccharide antigen was calculated based on batch volume and concentration of individual bulk polysaccharide concentration. The individual conjugate was added to a solution of histidine, sodium chloride and PS-20 to produce a 2-fold conjugate blend. The formulation vessel containing the 2-fold conjugate blend is mixed using a magnetic stir bar and then sterile filtered into another vessel. The sterile filtered, 2-fold conjugate blend is either added to another vessel containing Aluminum Phosphate Adjuvant (APA) or diluted with saline to achieve the desired target polysaccharide, excipient and APA (if required) concentrations. The formulations are then filled into glass vials or syringes and stored at 2-8° C.

### Example 9

### Impact of Conjugate/Formulation Processes on the Immunogenicity of 35B-CRM197 Vaccines

Young female CD1 mice (6-8 weeks old, n=5/group) were immunized with 0.1 ml of the 35B-CRM197 vaccine formulated above on day 0, day 14, and day 28. 35B-CRM197 vaccine was dosed at 0.4 μg of 35B polysaccharide conjugated to CRM197 without APA adjuvant (groups 1 to 6) or with 25 μg APA (groups 7 and 8) per immunization (Tables 7 and 8). Sera were collected prior to study start (pre-immune) and on day 35 (post-dose 3, PD3). Mice were observed at least daily by trained animal care staff for any signs of illness or distress. The vaccine formulations in mice were deemed to be safe and well tolerated, as no vaccine-related adverse events were noted. All animal experiments were performed in strict accordance with the recommendations in the Guide for Care and Use of Laboratory Animals of the National Institutes of Health. The mouse experimental protocol was approved by the Institutional Animal Care and Use Committee at Merck & Co., Inc.

Mice sera were evaluated for anti-PnPs35B IgG titers using ELISA as previously described (Chen Z. F. et al, BMC Infectious Disease, 2018, 18: 613) and anti-35B functional antibody through opsonophagocytosis assays (OPA) based on previously described protocols at www.vaccine.uab.edu and Opsotiter® 3 software owned by and licensed from University of Alabama (UAB) Research Foundation (Burton, R L, Nahm M H, Clin Vaccine Immunol 2006, 13:1004-9; Burton, R L, Nahm M H, Clin Vaccine Immunol 2012, 19:835-41). Pre-immune sera were assayed as a pool and PD3 sera were assayed individually.

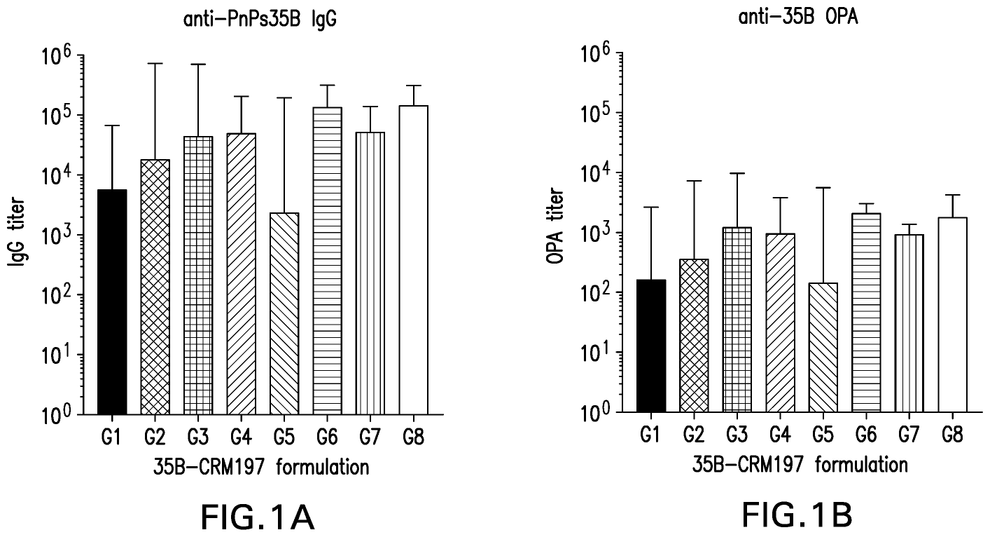

There was no detectable anti-35B IgG at 1:200 dilution for the pre-immune sera (data not shown). However, IgG titers increased at PD3 for all vaccines formulation (FIG. 1A). The data also demonstrate that different conjugation/formulation processes had significant impact on the immunogenicity of 35B polysaccharide-CRM197 vaccines. Adding APA adjuvant increases the IgG titers compared to vaccine without adjuvant (group 7 vs. 1/group 8 vs. 4). Adding 5 mM of NaCl to the conjugate reaction also increased the IgG titer of the 35B-CRM197 vaccine (group 6 vs. 5). The anti-35B OPA titers followed the same trend as seen in the IgG titer (FIG. 1B).

Serotype 35B polysaccharide-CRM197 conjugates with attributes across the ranges as shown in Table 7 were found to be immunogenic.

1. A serotype 35B S. pneumoniae polysaccharide-protein conjugate comprising a serotype 35B S. pneumoniae polysaccharide conjugated to a protein, the conjugate having a molecular weight of 1,000 kDa to 7,000 kDa.

2. The conjugate of claim 1 wherein the conjugate has a lysine consumption of 3 mol/mol protein to 9 mol/mol protein.

3. The conjugate of claim 1 wherein the conjugate has a lysine consumption of 4 mol/mol protein to 8 mol/mol protein.

4. A composition comprising the conjugate of claim 2, wherein the composition further comprises free polysaccharide of less than 30% of the total polysaccharide amount and free protein of less than 30% of the total protein amount.

5. A composition comprising the conjugate of claim 2, wherein the composition further comprises free polysaccharide of less than 20% of the total polysaccharide amount and free protein of less than 20% of the total protein amount.

6. The conjugate of claim 1, wherein the protein is CRM197.

7. The composition of claim 4, wherein the protein component of the polysaccharide-protein conjugate is CRM197.

8. A process for making the conjugate of claim 1 which comprises activation of the polysaccharide, wherein the activation utilizes periodate in a range of 0.01 to 0.1 moles of periodate per mole of polysaccharide repeating unit.

9. The process of claim 8 wherein the range of periodate is 0.03 to 0.06 moles of periodate per mole of polysaccharide repeating unit.

10. The process of claim 8 wherein the periodate is sodium periodate.

11. The process of claim 8 wherein the periodate is sodium metaperiodate.

12. A process for making the conjugate of claim 1 which comprises conjugating the polysaccharide to the protein, wherein the conjugation is performed at a conjugation temperature of between 22° C. to 38° C.

13. The process of claim 12 wherein the conjugation temperature is between 32° C. to 36° C.

14. A process for making the conjugate of claim 1 which comprises activation of the polysaccharide, wherein the activation utilizes periodate in a range of 0.01 to 0.1 moles of periodate per mole of polysaccharide repeating unit, and conjugating the polysaccharide to the protein, wherein the conjugation is performed at a conjugation temperature of between 22° C. to 38° C.

15. A process for making the conjugate of claim 1 which comprises activation of the polysaccharide, wherein the activation utilizes periodate in a range of 0.03 to 0.06 moles of periodate per mole of polysaccharide repeating unit, and conjugating the polysaccharide to the protein, wherein the conjugation is performed at a conjugation temperature of between 32° C. to 36° C.

16. The process of claim 14, wherein the conjugation is performed in an aprotic solvent.

17. The process of claim 16, wherein the aprotic solvent is DMSO.

18. The process of claim 16, wherein the conjugation is performed in the presence of sodium chloride.

19. The process of claim 18, wherein the concentration of sodium chloride is 5 to 15 mM.

20. The process of any of claim 16, wherein said solvent contains less than 1.2% water (v/v).

21. The process of claim 20, wherein the solvent contains less than 0.6% water (v/v).

22. The process of claim 21, wherein the solvent contains less than 0.3% water (v/v).

23. The process of claim 12, wherein the conjugation is performed with activated polysaccharide comprising an aldehyde per repeating unit in the range of 0.01 to 0.1.

24. The process of claim 23, wherein the aldehyde per repeating unit is in the range of 0.03 to 0.06.

25. The process of claim 12, wherein the conjugation is performed with activated polysaccharide with a molecular weight in the range of 30 to 200 KDa.

26. The process of claim 25, wherein the molecular weight range of the activated polysaccharide is 40 to 100 KDa.

27. An immunogenic multivalent pneumococcal conjugate vaccine composition comprising a serotype 35B S. pneumoniae polysaccharide-protein conjugate prepared by the process of claim 8.

28. An immunogenic multivalent pneumococcal conjugate vaccine composition comprising the serotype 35B S. pneumoniae polysaccharide-protein conjugate of claim 1.
